(19) United States
(12) Patent Application Publication
(10) Pub. No.: US 2005/0149021 A1
(43) Pub. Date: Jul. 7, 2005

(54) Spinal implant device

(76) Inventor: James E. Tozzi, Bethesda, MD (US)

Correspondence Address: JACOBSON HOLMAN PLLC, 400 SEVENTH STREET N.W., SUITE 600, WASHINGTON, DC, 20004, US

(21) Appl. No.: 11/019,262

(22) Filed: Dec. 23, 2004

Related U.S. Application Data
(60) Provisional application No. 60/531,684, filed on Dec. 23, 2003.

(51) Int. Cl. A61B 17/56

(52) U.S. Cl. 606/61

(57) ABSTRACT

A spinal implant device having an anatomical shape designed to mimic and restore normal human spinal anatomy. The devices comes in a range of sizes and are structured to specifically accommodate the structure of at least one of the cervical, thoracic and lumbar regions of the spine. Once affixed to the vertebrae, the devices may be used to effectively fuse two or more vertebrae, or to stabilize the vertebrae and protect the posterior portions of the spinal cord. Adjustments for sagittal plane contouring may also be effected through cable tensioning via spinous process fixation.

This application is entitled to and hereby claims the priority of co-pending U.S. Provisional application Ser. No. 60/531,684 filed Dec. 23, 2003.

### BACKGROUND OF THE INVENTION

1. Field of the Invention

The present invention is related to the field of spinal surgery and, more particularly, to a device for restoring vertebral anatomy at the time of laminectomy or for revision posterior spinal surgery.

2. Description of the Related Art

Following spinal fusion surgery, patients often experience difficulty due to post laminectomy instability, decompensated lumbar lordosis, transitional syndrome and “fusion disease”. Accordingly, a need exists for a device that is able to restore vertebral anatomy at the time of laminectomy or for revision posterior spinal surgery.

### SUMMARY OF THE INVENTION

In view of the foregoing, one object of the present invention is to overcome the difficulties associated with spinal fusion including post laminectomy instability, decompensated lumbar lordosis, transitional syndrome and “fusion disease”.

Another object of the present invention is to provide a spinal fusion implant device that restores vertebral anatomy at the time of laminectomy or for revision posterior spinal surgery.

A further object of the present invention is to provide a spinal implant device that allows for neural tissue protection and preservation of myofacial integrity via spinal muscle attachment and ligament anchoring.

An additional object of the present invention is to provide a spinal implant device for fusing two or more vertebrae.

Yet another object of the present invention is to provide a spinal fusion implant device in which adjustments for sagittal plane contouring may be effected through cable tensioning via spinous process fixation.

A further object of the present invention is to provide an assortment of spinal implant devices of varying sizes and shapes to suit different anthropomorphic dimensions and to accommodate vertebrae in the cervical, thoracic and lumbar regions.

A still further object of the present invention is to provide a spinal implant device having suture anchor holes to secure muscle and ligament tissue thereto for improved spinal alignment.

Another object of the present invention is to provide a spinal implant device for restoring posterior elements of the vertebrae and thereby act as a protective covering for the spinal cord and nerve roots.

It is yet another object of the invention to provide a spinal implant device that is not complex in structure and which can be manufactured at low cost but yet more effectively stabilize one or more spinal vertebrae at the time of laminectomy or for revision posterior spinal surgery.

In accordance with these and other objects, the present invention is directed to a spinal implant device for stabilizing a vertebrae or for fusing two or more vertebrae, the device being constructed in a number of sizes and shapes to accommodate the particular anatomical structure typical of the cervical, thoracic and lumbar regions of the spine, as well as the different sizes of each depending upon the patient. The device includes a plate-like body having a generally planar anterior surface and a corresponding posterior surface from which a projection extends outwardly. The body typically includes a plurality of apertures located in distal portions thereof for receiving fastening members, and the posterior projection is provided with a plurality of suture holes for anchoring muscles and ligaments to the posterior projection. Two-aperture devices, which may have a generally rectangular or a generally triangular anterior surface, are secured to opposing posterior surfaces of a single vertebrae so as to cover and protect the spinal cord and nerve roots, while four-aperture devices are typically rectangular in overall shape of the plate-like body portion, and may be used to connect two adjacent vertebrae to effect fusion thereof.

These together with other objects and advantages which will become subsequently apparent reside in the details of construction and operation as more fully hereinafter described, reference being had to the accompanying drawings forming a part hereof, wherein like numerals refer to like parts throughout.

### BRIEF DESCRIPTION OF THE DRAWINGS

FIG. 1 shows a posterior view of a two-vertebrae spinal implant fusion device as secured to two vertebrae, in accordance with the present invention;

FIG. 2 is a side view of the two-vertebrae spinal implant fusion device of FIG. 1;

FIG. 3 is a posterior view of the two-vertebrae spinal implant fusion device having variable-position fastening apertures;

FIG. 4 is a top view of the device of FIG. 3;

FIG. 5 is a posterior view of a single-vertebrae embodiment of the spinal implant device for thoracic and lumber vertebrae according to the present invention;

FIG. 6 is a side view of the device of FIG. 5;

FIG. 7 is a top view of the device of FIG. 5;

FIG. 8 is a posterior view of a single-vertebrae spinal implant device for cervical vertebrae, according to the present invention;

FIG. 9 is a side view of the device of FIG. 8;

FIG. 10 is a top view of the device of FIG. 8;

FIG. 11 is a top view of a variation of the cervical spinal implant device of FIG. 8, as secured to at least one cervical vertebrae;

FIG. 12 is another top view of the device of FIG. 11;

FIG. 13 is a posterior view of a four-aperture device for cervical vertebrae in accordance with the present invention;

FIG. 14 is a top view of another embodiment of a spinal implant device as secured to at least one thoracic vertebrae, in accordance with the present invention;

FIG. 15 is a posterior view of a two-vertebrae spinal fusion implant device with variable-position fastening apertures and three suture holes according to the present invention; and

FIG. 16 is a side view of the device of FIG. 15.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar purpose.

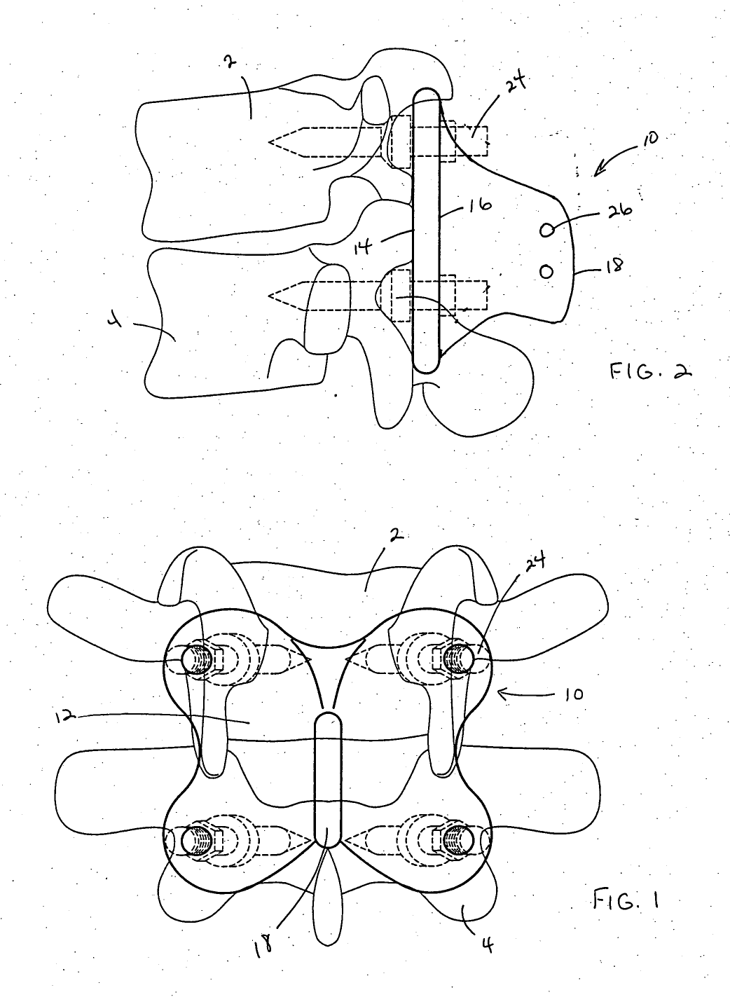

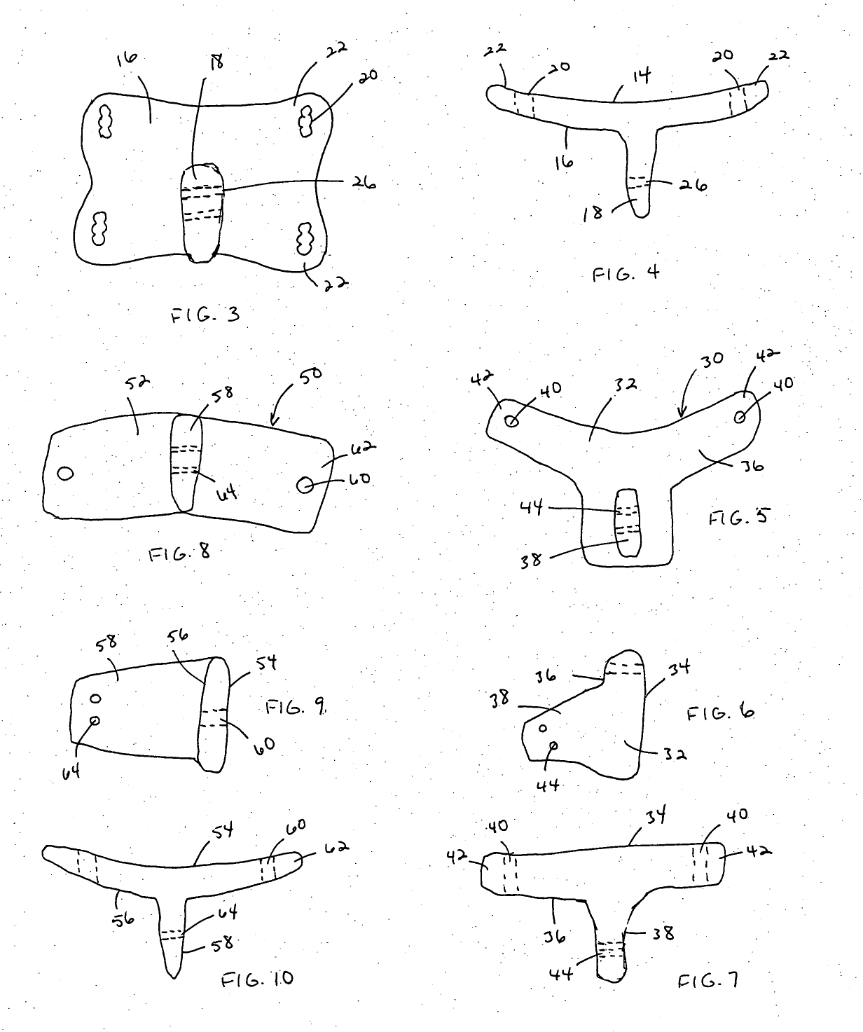

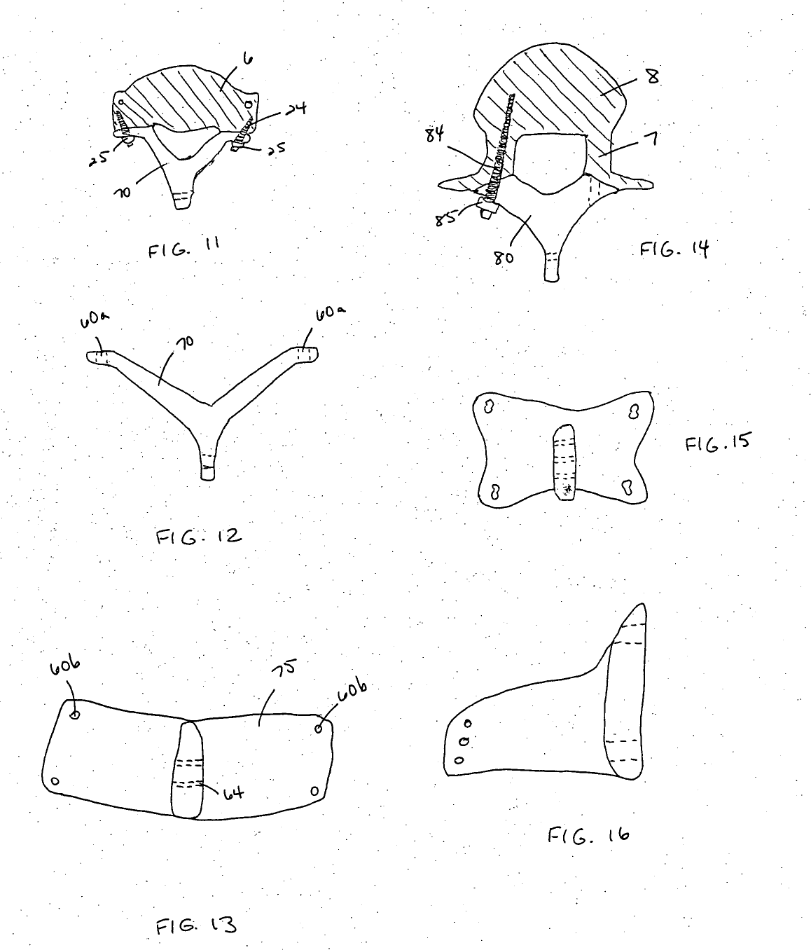

As representatively illustrated in FIGS. 1-4, according to a first embodiment the present invention is directed to a spinal implant device, generally designated by the reference numeral 10, with a body 12 having a generally planar anterior surface 14 and a corresponding posterior surface 16 from which a projection 18 extends outwardly, the projection being substantially perpendicular to the posterior surface. The planar body 12 has a generally rectangular shape when viewed from the anterior or posterior sides, and typically includes a plurality of through-passing apertures 20 located in distal or corner portions 22 thereof for receiving fastening members 24. The apertures may provide for variable positioning of the fastening members as shown in FIG. 3, or may be generally circular for a single position. The posterior projection 18 is provided with a plurality of suture holes 26 for anchoring muscles and ligaments to the posterior projection 18.

As shown, the embodiment set forth in FIGS. 1-4 has four apertures through which four fastening members may be inserted. Fastening members through the upper two apertures are secured to a first vertebrae 2, and fastening members through the lower two apertures are secured to a second vertebrae 4. When so secured, the first and second vertebrae 2, 4 are effectively fused together.

A second embodiment of the spinal implant device according to the present invention, generally designated by the reference numeral 30, is illustrated in FIGS. 5-7. As with the previous embodiments, the device 30 includes a body 32 having a generally planar or gently curved anterior surface 34 and a corresponding posterior surface 36 from which a projection 38 extends outwardly. When viewed from the anterior or posterior sides, the body 32 has a somewhat triangular shape and typically includes at least two through-passing apertures 40 located in distal portions 42 thereof for receiving fastening members (not shown). The posterior projection 38 is provided with a plurality of suture holes 44 for anchoring muscles and ligaments to the posterior projection 38.

The first and second embodiments are designed for lumbar and thoracic vertebrae, with the four-aperture embodiment of FIGS. 1-4 being used to fuse two vertebrae and the dual-aperture embodiment of FIGS. 5-7 serving to stabilize a single vertebrae and cover the posterior portion of the spinal cord after the original bone has been removed from the affected vertebrae during surgery or as a result of injury or disease.

A third embodiment of the spinal implant device according to the present invention, generally designated by the reference numeral 50, is illustrated in FIGS. 8-10 and is particularly suited for use with cervical vertebrae. As with the previous embodiments, the device 50 includes a body 52 having a generally planar anterior surface 54 and a corresponding posterior surface 56 from which a projection 58 extends outwardly, generally perpendicular to the posterior surface 56. When viewed from the anterior or posterior surfaces, the body 52 has a generally rectangular shape and typically includes a plurality of apertures 60 located in distal portions 62 thereof for receiving fastening members (not shown). The posterior projection 58 is provided with a plurality of suture holes 64 for anchoring muscles and ligaments to the posterior projection 58.

In each of the three embodiments, the posterior projection has a shape intended to replicate the spinous process of corresponding vertebrae from which the spinous process has been removed, with the design in each case of the device making it particularly suitable for the corresponding vertebrae in a particular spinal region. Cervical, thoracic, and lumbar implant devices are sized and shaped based upon anthropomorphic dimensions, with each subset of implants being further divided into sizes based upon individual variation in spino-laminar height and width. The devices are preferably made of titanium, although other metals such as stainless steel could also be used.

In the neck or cervical spine, the device is used to either fuse two or more vertebrae or to restore the posterior elements, thus acting as a protective covering of the spinal cord and nerve roots as well as an anchoring device for the muscles and ligaments of the posterior aspect of the neck. The device is secured via lateral mass fixation but, pending FDA approval, will ultimately be transfixed via pedicle screws in a manner known in the art. The two-hole device shown in FIGS. 8-10 is used for single level fixation or one vertebrae.

FIGS. 11 and 12 illustrate a variation of the embodiment shown in FIG. 8, with the device 70 shown as mounted and unmounted, respectively, with respect to a cervical vertebrae. As shown in FIG. 11, the anterior surface is more greatly curved, having foot portions for contact with the vertebrae through which apertures 60a are provided. Fastening elements 24 are placed through the apertures 60a into the bone 6 of the lateral mass of the cervical spine, where the fastening elements 24 are then anchored firmly to the device with a locking nut 25 on top of the body 70, thus achieving fixation of the device to the vertebrae. A further variation is shown in FIG. 13, depicting a device 75 having four apertures 60b.

To accomplish spinal fusion of two or more vertebrae, a four-aperture cervical plate of the type shown in FIGS. 1-4 is used. A fastening member such as a pedicle screw is placed through each of the four apertures and secured with a locking nut. Due to the resilience of the disk space located anterior to the spinal cord, the device can transmit angular adjustments to the vertebrae via cable tensioning of the posterior projection at the time of muscle and ligament fixation, thus achieving correction of spinal alignment in the sagittal plane. The attachment of soft tissues, muscles and/or ligaments to the posterior projection 18, 38, 58, is afforded by the suture holes as illustrated.

In order to maintain motion between adjacent vertebrae, a plate with two holes such as that shown in FIGS. 8-10 is anchored to a single vertebrae and the posterior spinal ligaments and muscles are attached to the posterior projection of the device and tensioned according to the need to adjust the spinal alignment in the sagittal plane. Through proper tensioning and positioning, spinal dynamics and muscle function are improved.

Similarly, the device may be applied to the thoracic and lumbar spine in the same manner, being mounted on pedicle screws fastened to each vertebrae and secured with locking nuts, as shown by device 80 in FIG. 14. Screws 84 are placed into the pedicle region 7 on each side of the vertebrae 8 so as to extend through 50%-80% of the vertebral body, with a protruding portion of the screws having a length of about 7-12 mm. The device is placed over these extending portions of the screws 84 and locked into position using a securing mechanism such as a locking nut 85. Alternatively, sublaminar hooks (not shown) may be used for laminar, transverse process fixation to secure the device to the thoracic and lumbar vertebrae.

Any of the devices may have any number of suture holes provided in the posterior projection, such as three holes in the four-aperture thoracic and lumbar spinal implant device shown in FIGS. 15 and 16. The holes are formed in the projection during manufacture thereof.

As described, the present invention provides for the restoration of spinal anatomy, preventing spinal instability and malalignment arising from loss of bone in the sagittal plane. The device also protects the neural elements and enables proper spinal alignment to be maintained through the attachment of ligaments to the device. Problems often associated with loss of the spinous process, such as flat back, are therefore avoided and the likelihood of normal patient activity and function is enhanced.

The foregoing descriptions and drawings should be considered as illustrative only of the principles of the invention. The invention may be configured in a variety of shapes and sizes and is not limited by the dimensions of the preferred embodiments. Numerous applications of the present invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

1. A spinal implant device comprising:
a body having an anterior surface and a posterior surface;
a projection extending outwardly from a center portion of said posterior surface so as to be generally perpendicular to said center portion; and
a plurality of apertures in said body for receiving fastening members to secure said device to at least one vertebrae.

2. The spinal implant device according to claim 1, wherein said projection includes a plurality of suture holes for anchoring muscles and/or ligaments to said projection.

3. The spinal implant device according to claim 1, wherein said body is generally rectangular having four corners, each of said corners being provided with one of said plurality of apertures passing therethrough.

4. The spinal implant device according to claim 3, wherein said anterior surface is generally planar.

5. The spinal implant device according to claim 1, wherein said body is generally rectangular having two shorter sides and two longer sides, each of said shorter sides being provided with one of said plurality of apertures passing therethrough.

6. The spinal implant device according to claim 1, wherein said body is generally T-shaped, having a horizontally extending bar portion generally centered over a vertically extending stem portion.

7. The spinal implant device according to claim 6, wherein said anterior surface is generally planar.

8. The spinal implant device according to claim 6, wherein said bar portion is provided at each end with one of said plurality of apertures, and said projection extends from said stem portion.

9. The spinal implant device according to claim 6, wherein said anterior surface is curved and said body includes two foot portions for contacting the vertebrae, each foot being provided with one of said plurality of apertures.

10. The spinal implant device according to claim 1, wherein said apertures are generally circular.

11. The spinal implant device according to claim 3, wherein said apertures have a slotted configuration for variable positioning of fastening members in each corner.

12. The spinal implant device according to claim 1, wherein a shape of said projection replicates a spinous process of a corresponding vertebrae.

13. A spinal implant device for restoring vertebral anatomy and/or stabilizing a vertebrae, comprising:
a body having generally parallel anterior and posterior surfaces;
a projection extending outwardly from a center portion of said posterior surface so as to be generally perpendicular to said center portion, said projection having a shape replicating a spinous process of a corresponding vertebrae being restored or stabilized;
a plurality of apertures in said body for receiving fastening members to secure said device to a vertebrae; and
a plurality of suture holes in said projection for anchoring muscles and/or ligaments thereto.

14. The spinal implant device according to claim 13, wherein said body is generally rectangular with each corner provided with one of said plurality of apertures passing therethrough.

15. The spinal implant device according to claim 13, wherein said body is generally rectangular having two shorter sides and two longer sides, each of said shorter sides having one of said plurality of apertures passing therethrough.

16. The spinal implant device according to claim 13, wherein said body is generally T-shaped, having a horizontally extending bar portion generally centered over a vertically extending stem portion.

17. The spinal implant device according to claim 16, wherein each end of said bar portion has one of said plurality of apertures passing therethrough, and said projection extends from said stem portion.

18. The spinal implant device according to claim 16, wherein said anterior surface is curved and said body includes two foot portions for contacting the vertebrae, each foot being provided with one of said plurality of apertures.

19. The spinal implant device according to claim 13, wherein said apertures have a slotted configuration for variable positioning of fastening members.

20. A spinal implant device comprising a body having anterior surface and posterior surfaces, and a projection extending outwardly from a center portion of said posterior surface so as to be generally perpendicular to said center portion, said body being structured to specifically accommodate at least one of cervical, thoracic and lumbar regions of a human spine.
